(19) United States
(12) Patent Application Publication
(10) Pub. No.: US 2018/0217124 A1
(43) Pub. Date: Aug. 2, 2018

(54) MODEL FOR ASSESSMENT OF KIDNEY FUNCTION IN CATS AND EVALUATION OF RELATED TREATMENT PROTOCOLS

(71) Applicant: Ramp Research, LLC, The Woodlands, TX (US)

(72) Inventor: Patrick J. Casey, Kumeu (NZ)

(21) Appl. No.: 15/884,183

(22) Filed: Jan. 30, 2018

Related U.S. Application Data
(60) Provisional application No. 62/452,086, filed on Jan. 30, 2017.
(60) Provisional application No. 62/468,724, filed on Mar. 8, 2017.

(51) Int. Cl. G01N 33/49; G01N 33/50

(52) U.S. Cl. CPC G01N 33/49; G01N 2333/46; G01N 2800/347; G01N 33/5088

(57) ABSTRACT

The invention features a method for a model for a determination of an evolution of a level of kidney function over time in a cat. The invention also features a method of assessing an effectiveness of a treatment protocol for affecting kidney function in a cat.

### STATEMENT REGARDING FEDERALLY SPONSORED RESEARCH OR DEVELOPMENT

Not Applicable

### BACKGROUND OF THE INVENTION

The present invention broadly relates to the assessment of kidney function in mammals, and in particular to models for the measurement of kidney function in cats. Such models can then be used for the development of treatment protocols for cats and other mammals including humans.

Much attention has been paid to the onset and progression of chronic kidney disease in mammalian species. Some commentators consider that not a lot of progress has been achieved in what is seen broadly as a progressive and minimally responsive condition that often can lead to patient death. De Nicola L, Provenzano M, Chiodini P, Borrelli S, Russo L, Bellasi A, Santoro D, Conte G, Minutolo R (2017). Epidemiology of low-proteinuric chronic kidney disease in renal clinics. PLoS One. 2017 February 17; 12(2): eCollection 2017. One potential reason for the lack of progress is the lack of a good quantifiable model for the assessment of progressive kidney disease. To date most scientific attention has focused on the hypoxic ischemic model which in itself has some experimental limitations due to its nature of induction. Li G, Zhang H X, Wang Y P, Zhang J, Hong K, Tian X J, Ma L L (2015) Protective effect of phloroglucinol on renal ischemia and reperfusion injury].[Article in Chinese]. Beijing Da Xue Xue Bao. Oct. 18; 47(5):743-8. Although it is known that cats suffer from kidney dysfunction similarly to humans, there is no known model for assessing the decrease in kidney function over a finite time period in cats.

### BRIEF SUMMARY OF THE INVENTION

The present invention features a chronic cat model as a model which may be utilized for the assessment of progressive kidney disease and for the evaluation of treatment modalities for kidney dysfunction. The glomerular filtration rate (“GFR”) directly measures filtration of the kidney. The GFR is calculated based on the level of a GFR indicator such as, for a non-limiting example, iohexol plasma elimination or clearance, and provides an indication of kidney function. The GFR typically decreases in mammals with age, disease, trauma, or other abnormalities. Decreased GFR indicates that the kidneys are removing fewer waste products from the blood stream.

In one aspect, the invention features a method for a model for a determination of an evolution of a level of kidney function over time in a cat. The method includes sampling a first level of a GFR indicator of the cat at a first point in time and calculating a baseline GFR based on the first level of the GFR indicator; sampling at least one second level of the GFR indicator of the cat at at least one second point in time and calculating at least one second GFR based on the at least one second level of the GFR indicator; determining the evolution of the level of the kidney function of the cat over time based on a difference between the baseline GFR and the at least one second GFR; wherein a time range between the first point in time and the second point in time is in a range of 30 to 120 days; wherein the baseline GFR of the cat is less than the baseline GFR of a healthy cat of a same gender; and correlating the evolution of the level of the kidney function over time with a corresponding measurement of apoptosis of the glomeruli of the cat over time.

In one embodiment, the time range between the first point in time and the at least one second point is a minimum of approximately 60 days.

In one embodiment, sampling the first level of the GFR indicator at the first point in time includes: administering a dose having an initial concentration of a contrast agent to the cat; after a select first time interval, at the first point in time, taking a first blood sample of the cat and measuring a first subsequent concentration of the contrast agent in the first blood sample of the cat; determining a first level of elimination of the contrast agent in the first blood sample of the cat based on a first difference between the initial concentration of the contrast agent and the first subsequent concentration of contrast agent in the first blood sample of the cat; and correlating the first level of elimination of the contrast agent in the first blood sample of the cat with the first level of the GFR indicator at the first point in time.

In one embodiment, sampling at least one second level of the GFR indicator of the cat at the at least one second point in time includes: after at least one select second time interval, at the at least one second point in time, taking at least one second blood sample of the cat and measuring at least one second subsequent concentration of the contrast agent in the at least one second blood sample of the cat; determining at least one second level of elimination of the contrast agent in the at least one second blood sample of the cat based on at least one second difference between the first subsequent concentration of the contrast agent in the first blood sample of the cat and the at least one second subsequent concentration of the contrast agent in the at least one second blood sample of the cat; and correlating the at least one second level of elimination of the contrast agent in the at least one second blood sample of the cat with at least one second level of the GFR indicator at the second point in time.

In one embodiment, the contrast agent includes an iodinated, non-ionic, radio contrast agent. In another embodiment, the contrast agent includes iohexol.

In different embodiments, the dose of the contrast agent has an initial concentration in a range of about 0.5 mL/Kg to about 4.5 mL/Kg of body weight of the cat, and preferably in a range of 1 mL/Kg of body weight to about 2 mL/Kg of body weight of the cat.

In one embodiment, the contrast agent includes a diluting agent. In a preferred embodiment, the diluting agent includes a saline solution.

In one embodiment, the GFR indicator includes plasma clearance of iohexol.

In another aspect, the invention features a method of assessing an effectiveness of a treatment protocol for affecting kidney function in a cat. The method includes: providing a plurality of cats for a control group and a plurality of cats for a treatment group; at a first point in time, measuring a first level of a GFR indicator and calculating a baseline GFR based on the first level of the GFR indicator in each of the plurality of cats in the control group and in each of the plurality of cats in the treatment group; pairing each of the plurality of cats in the treatment group with a corresponding cat in the plurality of cats in the control group based on a substantial similarity in the baseline GFR; after calculation of the baseline GFR, administering to each of the plurality of cats in the treatment group a treatment composition according to the treatment protocol specifying a treatment dose and a treatment dosing interval; at a select assessment time period at at least one second point in time following administering the treatment composition, measuring at least one second level of the GFR indicator and calculating at least one second GFR based on at least one second level of the GFR indicator in each of the plurality of cats in the control group and in each of the plurality of cats in the treatment group; determining an evolution of a level of a kidney function over time based on a difference between the baseline GFR and the at least one second GFR over the select assessment time period for each of the plurality of cats in the control group and for each of the plurality of cats in the treatment group; assessing the effectiveness of the treatment protocol for affecting kidney function in the cat based on a comparison of the evolution of the level of the kidney function over time in each of the plurality of cats in the treatment group with the evolution of the level of the kidney function over time in the corresponding cat in the plurality of cats in the control group; wherein the baseline GFR of each of the plurality of cats in the treatment group and each of the plurality of cats in the control group is less than the baseline GFR of a healthy cat of a same gender.

In one embodiment of this method of the invention, the select assessment time period is in a range of 45 days to 120 days. In a preferred embodiment of the invention, the select assessment time period is a minimum of approximately 60 days.

In one embodiment, a lack of change in the evolution of the level of the kidney function over time in the cat is correlated with a decrease in death of a plurality of cells in a glomerulus of at least one kidney in the cat.

In one embodiment, a positive change in the evolution of the level of the kidney function over time in the cat is correlated with a positive change in functionality of a plurality of cells in a glomerulus of at least one kidney in the cat.

In one embodiment, the method further includes identifying a treatment protocol for a mammal other than the cat based on an extrapolation to the mammal of the treatment protocol for affecting kidney function in the cat. In another embodiment, the mammal is a human.

In one embodiment, measuring the first level of the GFR indicator at the first point in time includes: administering a dose having an initial concentration of a contrast agent to each of the plurality of cats in the control group and in the treatment group; after a select first time interval, at the first point in time, taking a first blood sample and measuring a first subsequent concentration of the contrast agent in the first blood sample of each of the plurality of cats in the control group and in the treatment group; determining a first level of elimination of the contrast agent in the first blood sample based on a first difference between the initial concentration of the contrast agent and the first subsequent concentration of the contrast agent in the first blood sample of each of the plurality of cats in the control group and in the treatment group; and correlating the first level of elimination of the contrast agent in the first blood sample with the first level of the GFR indicator at the first point in time in each of the plurality of cats in the control group and in the treatment group.

In another embodiment, measuring the at least one second level of the GFR indicator at the at least one second point in time includes: after the at least one select second time interval, at the at least one second point in time, taking at least one second blood sample and measuring at least one second subsequent concentration of the contrast agent in the at least one second blood sample of each of the plurality of cats of the control group and of the treatment group; determining at least one second level of elimination of the contrast agent in the at least one second blood sample based on at least one second difference between the first subsequent concentration of the contrast agent in the first blood sample and the at least one second subsequent concentration of the contrast agent in the at least one second blood sample of each of the plurality of cats in the control group and in the treatment group; and correlating the at least one second level of elimination of the contrast agent in the at least one second blood sample with the at least one second level of the GFR indicator at the second point in time in each of the plurality of cats in the control group and in the treatment group.

In one embodiment, the contrast agent includes an iodinated, non-ionic radio contrast agent. In a preferred embodiment, the contrast agent includes iohexol.

In different embodiments, the dose of the contrast agent has an initial concentration in a range of about 0.5 mL/Kg to about 4.5 mL/Kg of body weight of the cat, and preferably in a range of 1 mL/Kg of body weight to about 2 mL/Kg of body weight of the cat.

In one embodiment, the GFR indicator includes plasma clearance of iohexol.

### BRIEF DESCRIPTION OF THE SEVERAL VIEWS OF THE DRAWINGS

These and other advantageous aspects of the invention will be evident from the following detailed description, which should be considered in conjunction with the attached drawings, wherein:

FIG. 1 is a graph showing a treatment timeline of male and female cats treated according to a model of one embodiment of the invention;

FIG. 2 is a graph showing the level of GFR over time for control, untreated male cats according to a model of one embodiment of the invention;

FIG. 3 is a graph showing the level of GFR over time for control, untreated female cats according to a model of one embodiment of the invention;

FIG. 4 is a graph showing the effect on GFR over time for male cats treated according to a model of one embodiment of the invention;

FIG. 5 is a graph showing the effect on GFR over time for female cats treated according to a model of one embodiment of the invention;

FIG. 6 is a graph showing the level of HSP 70 over time for control, untreated male and female cats, according to a model of one embodiment of the invention;

FIG. 7 is a graph showing the level of HSP70 over time in male and female cats treated according to a model of one embodiment of the invention;

FIG. 8 is a graph showing the level of cell free DNA or cfDNA over time in male and female cats treated according to a model of one embodiment of the invention; and

FIG. 9 is a graph showing a relationship between GFR, cellular necrosis, apoptosis and anastasis in subjects according to a model of one embodiment of the invention.

### DETAILED DESCRIPTION OF THE INVENTION

This patent application claims priority from U.S. Provisional Patent Application No. 62/452,08, filed Jan. 30, 2017, and U.S. Provisional Patent Application No. 62/468,724, filed Mar. 8, 2017, the disclosures of which are incorporated by reference herein in their entirety.

The present invention features a model for assessing kidney function over time in cats. The model is characterized by a determination of the evolution of a level of the cat's kidney function or efficiency of performance over a finite time period. The function of the cat's kidney at a particular point in time is shown by a measurement of the GFR of the cat which in turn is calculated based on the level of a GFR indicator. The GFR indicator can include a measurement of a level of the elimination or clearance of a contrast agent from the blood or plasma of the cat. The contrast agent can include, for a non-limiting example, an iodinated or otherwise dyed non-ionic radio contrast agent. A non-limiting example of a contrast agent suitable for the present invention includes iohexol.

The contrast agent can be administered to the subject cat using methods known to those of ordinary skill in the art. In one embodiment, the dose is administered to the cat using an injection procedure. In a preferred embodiment, the contrast agent is administered using a subcutaneous injection procedure. The dose of the contrast agent can have an initial concentration in a range of about 0.5 mL/Kg to about 4.5 mL/Kg of body weight of the cat, and preferably in a range of 1 mL/Kg of body weight to about 2 mL/Kg of body weight of the cat.

The contrast agent can include a diluting agent such as, for a non-limiting example, a saline solution.

The evolution of the level of the cat's kidney function is determined based upon a comparison of GFR measurements taken at the beginning and at the end of the assessment period(s). Naturally, multiple GFR measurements can be taken throughout the assessment period to obtain a more detailed picture of the progression or evolution of the kidney function in the subject cat.

It is surprisingly shown here that the GFR of cats which already exhibit a baseline GFR less than that of a comparable healthy cat of the same gender, will decrease measurably over a finite assessment time period of as little as 30 to 120 days and for purposes of the development of a measureable model, preferably over a finite time period of as little as 60 days.

The unique model of the invention can then be used for correlating the evolution of the level of the cat's kidney function over time with a corresponding measurement of necrosis, apoptosis and/or anastasis over time of the cells which make up glomeruli or capillary networks of the cat's kidneys. Necrosis is defined as, for purposes of the present application, “cellular death that occurs as the result of injury, disease and/or failure of blood supply to the cells”. Apoptosis is defined as, for the purposes of the present application, “cellular death that is programmed by the cell and occurs as a normal and controlled part of an organism's growth or development”. Anastasis is defined as, for the purposes of the present application, “cellular recovery from a debilitating condition”. Thus, the model can be used to measure necrosis or apoptosis, a slowing, prevention or reversal of apoptosis or necrosis, and anastasis in response to a particular treatment protocol, as shown in FIG. 9.

The unique model of the invention can also be used to assess the effectiveness of treatment protocols for affecting kidney function in cats. The same monitoring system could be used to evaluate the effectiveness of treatment in other mammalian species. The results of such effectiveness studies can then be extrapolated for the development of treatment protocols for other mammals. Notably, cats suffer kidney dysfunction similarly to humans. The model can be very important in, for example, the Human to show an increase in kidney functionality through GFR assessment. Thus, the model of the present invention can be extrapolated to develop treatment protocols for affecting kidney function in humans.

Acute and chronic kidney disease is characterized by the loss or death of cells which make up the glomeruli which process waste products in the blood stream. A lack of change in the evolution of kidney function over the assessment time period in the treated cats of the present invention can indicate a decrease in glomeruli cellular loss as a result of the treatment protocol. A positive change in the evolution of kidney function in the treated cats of the present invention over the assessment time period can indicate a positive change in the glomeruli cellular functionality as a result of the treatment protocol.

### Example 1

A total of 42 domestic cats including 21 male cats and 21 female cats were provided for study. The cats were selected for the study according to the following criteria:
minimum age of 8 years;
physical health as defined by routine physical examination;
stable body weight within normal season variation for a period of time of at least 6 months prior to the onset of the study;
absence of serious illness which might require euthanasia within 18 weeks of the study;
absence of hypothyroidism; and
absence of fractious behavior rendering the subject cat unsuitable for repeated collection of plasma samples.

The selected cats were housed in groups of 8-10 cats in indoor-outdoor purpose colony cages built at the Centre for Feline Nutrition Unit at Massey University, Palmerston North, New Zealand. The cats were fed a complete and balanced commercial high moisture low carbohydrate (canned) feline diet and had ad libitum access to water.

Two cats were deemed too fractious for sampling and were excluded after initial measurement of the GFR. Another cat died during the early part of the trial. The final allocation included 22 cats in the treatment population and 17 cats in the control population. The oldest and youngest cats studied were respectively 14.93 and 9.31 years of age while the mean age of the studied cats was 11.22 years.

The husbandry of the cats was compiled under the Massey University ethic committed protocol number 12/12. All studies were approved by the Animals Welfare and Ethics committee of Massey University, New Zealand.

The cats were initially divided into groups of 10 and the GFR of each cat was measured using the iohexol clearance method. The cats were then separated into a control group and a treatment group to provide a balanced distribution of the measured GFR in each group. Following the separation of the cats into 2 groups, there was no significant difference in the distribution of the measured GFR between the two groups. The trial commenced approximately 6 weeks later, maintaining a staggered approach, with 10 cats starting treatment each week for 4 weeks.

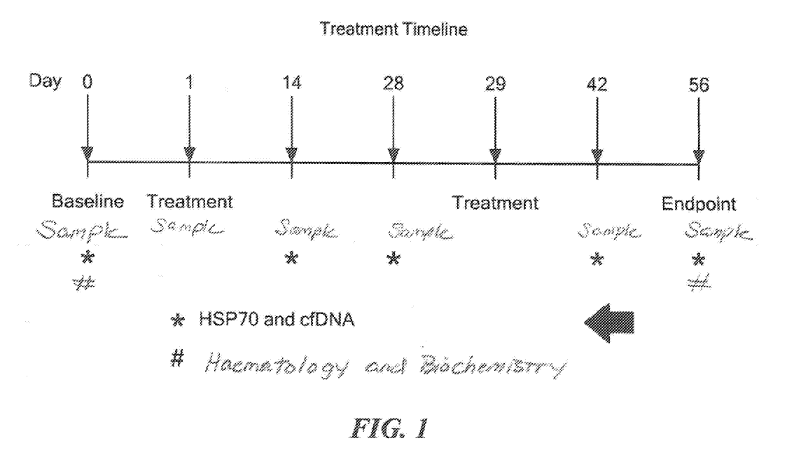

A venous blood sample of each cat was taken on day 0, 1, 14, 28, 42 and 56 of the study, as shown in FIG. 1. Measurements of GFR, Circulating Plasma HSP70 and cell free DNA were taken on days 0 and 56. Each cat was injected subcutaneously in the interscapular region with either a treatment or a control saline solution on days 1 and 29. The treatment consisted of 0.5 mL of product consisting of 250 mg lyophilized Chaperonze® reconstituted in 6 mL of saline. The control solution consisted of 0.5 mL saline. Control and treated cats were paired on the basis of their baseline GFR values. Injection sites were palpated for 1 week after each injection. All cats were weighed weekly during the trial period.

Blood Analysis

All blood samples were collected by jugular venipuncture and after application of 2% lignocaine gel, placed into Beckdin Dickinson blood collection tubes including the liquid form of Ethylenediaminetetraacetic acid or EDTA for circulating plasma harvesting. On days 0 and 56, a sample of EDTA blood, and clotted blood were submitted to the diagnostic laboratory for routine haematological and biochemical analysis. Blood placed into plain tubes for serum collection were allowed to clot at room temperature for 15-30 minutes and then centrifuged. Within 30 minutes of collection, EDTA plasma samples were centrifuged at 1300 rpm in a refrigerated centrifuge and plasma was separated. All serum and plasma samples were stored at −80° C. until transported for assaying.

GFR Measurement

The GFR was determined in each cat using the plasma clearance of iohexol (PCio) method as described by Miyamoto (2001). Miyamoto K (2001). Clinical application of plasma clearance of iohexol in feline patients. Feline Med Surg; 3(3):143-7. PubMed PMID: 1063. Plasma clearance of iohexol was determined after IV administration of Iohexol and plasma concentrations of iodine were measured by use of a colorimetric assay. The iohexol was administered using a single intravenous bolus injection in a dose of 1.5 mL of iohexol solution/Kg body weight of the cat. The iohexol solution contained Omnipaque 300, containing 647 mg of iohexol per milliliter equivalent to 300 mg of iodine per milliliter; Schering, Milan, Italy. Results for PCio were compared with simultaneously obtained values for urinary clearance of creatinine (CCr).

HSP70

Frozen plasma was transported to Endocrinology Labs, School of Veterinary Medicine, UC Davis, USA for HSP70 analysis. The sample dilution was 1:1 dilution, 200 μl buffer mixed with 200 μl serum, followed by the addition of 100 μl of the diluted serum to the wells. ELISA analysis was performed using High sensitivity ELISA Kit (Cat: ADI-EKS-715 Enzolifesciences, Farmingdale, N.Y. 11735), according to the manufacturer's instruction.

Cell Free DNA

The Qubit dsDNA BR (broad range) assay kit was used to determine cell free DNA concentration. The cell free DNA assays were performed using the general assay kits protocol, which utilizes a simple mix and read format for DNA. A number of 0.5 mL tubes were set up for standards and samples. The Qubit™ working solution was prepared by diluting the reagent 1:200 in Qubit™ protein buffer. The Qubit™ working solution was pipetted in an amount of 190 uL of into each of the tubes used for standards. An amount of 10 uL of each Qubit™ standard was added to the appropriate tube and vortex. The Qubit™ working solution was pipetted into individual assay tubes so that the final volume in each tube after adding sample was 200 uL. Each sample was pipetted into a corresponding assay tube and mixed. From the Home Screen of the Qubit® 2.0 Fluorometer, the Protein was selected and a new calibration was run. Upon the completion of the measurement, the result for each sample was read from the screen, removed from the Fluorometer, and the next sample inserted. This procedure was repeated until all the samples were read.

Results

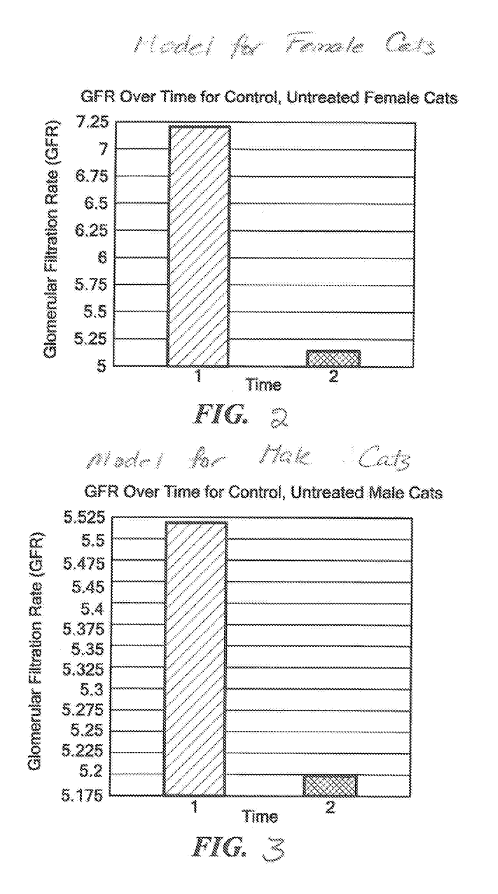

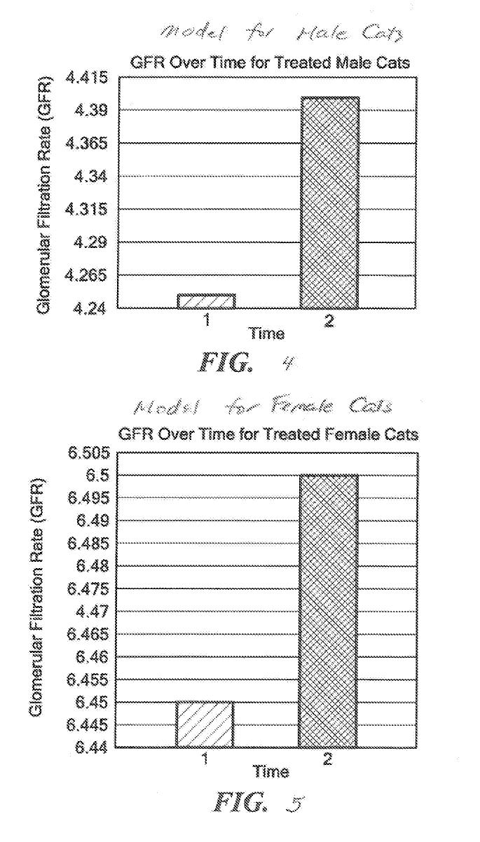

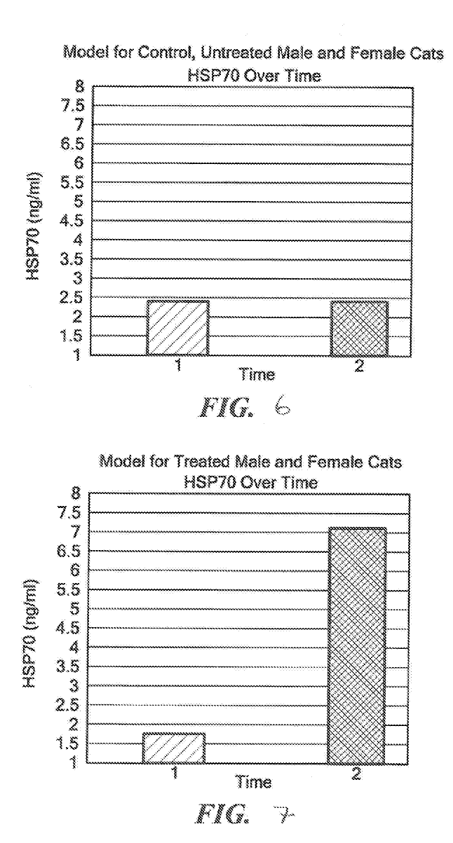

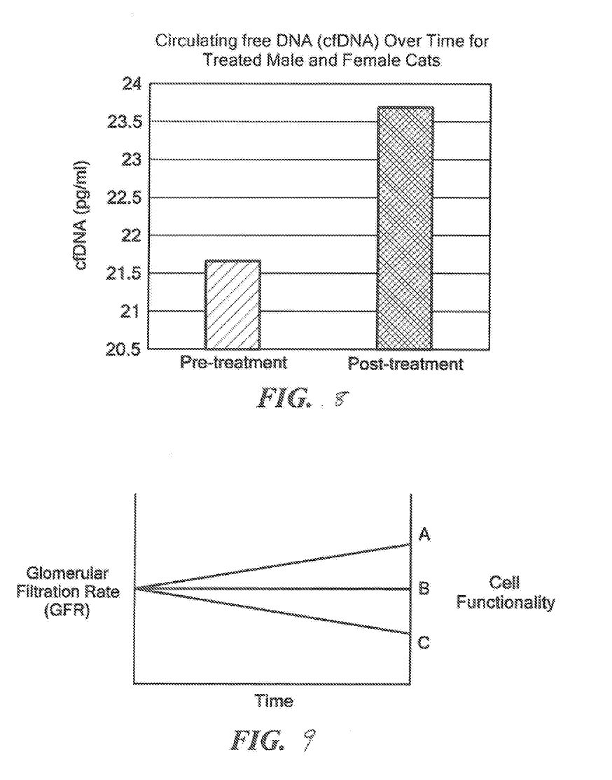

The control, untreated female and male cats showed a decreased GFR at the end of the assessment period, as shown in respective FIGS. 2-3. Treated male and female cats showed increased GFR at the end of the assessment period, as shown in respective FIGS. 4-5. Gender appeared to influence GFR both before and after treatment. FIG. 6 shows that control, untreated male and female cats had a substantially constant level of extracellular HSP70 over time, while the treated male and female cats had an increase in extracellular HSP70 over time and an increase in cell free DNA, as shown in respective FIGS. 7 and 8.

FIG. 9 graphically represents the progression of renal cell/organ function correlated to GFR measurement over time according to the model of the invention. For example, the natural decrease in GFR over time in the untreated, cats is correlated to a decrease in renal cell/organ function. The decrease in renal cell/organ function represents the normal progression of necrosis/apoptosis in the untreated cats, as shown by line “C” in FIG. 9. Successful treatment can possibly prevent the typical decrease in GFR over time in the treated cats. In such an example, the GFR in the treated cats would remain constant over time. The constant GFR would be correlated to the prevention of the expected decrease in renal cell/organ function, that is, the prevention of the expected progression of necrosis/apoptosis over time, as shown by line “B” in FIG. 9. In the example of the present invention, an increase in GFR over time was measured in the treated cats. Thus, the increase in GFR is correlated to a conversion of non-functional cells from the state of apoptosis to the state of anastasis, as shown by line A of FIG. 9.

The foregoing example and detailed description are not to be considered limiting of the invention which is defined by the following claims. The invention is understood to encompass such obvious modifications thereof as would be apparent to those of ordinary skill in the art.

What is claimed is:

1. A method for a model for a determination of an evolution of a level of kidney function over time in a cat comprising:
sampling a first level of a GFR indicator of the cat at a first point in time and calculating a baseline GFR based on the first level of the GFR indicator;
sampling at least one second level of the GFR indicator of the cat at at least one second point in time and calculating at least one second GFR based on the at least one second level of the GFR indicator;
determining the evolution of the level of the kidney function of the cat over time based on a difference between the baseline GFR and the at least one second GFR;
wherein a time range between the first point in time and the second point in time is in a range of 30 to 120 days;
wherein the baseline GFR of the cat is less than the baseline GFR of a healthy cat of a same gender; and
correlating the evolution of the level of the kidney function over time with a corresponding measurement of apoptosis over time of the glomeruli of the cat.

2. The method of claim 1 wherein the time range between the first point in time and the at least one second point is a minimum of approximately 60 days.

3. The method of claim 1, wherein sampling of a first level of the GFR indicator at the first point in time comprises:
administering a dose of a contrast agent having an initial concentration to the cat;
after a select first time interval, at the first point in time, taking a first blood sample of the cat and measuring a first subsequent concentration of the contrast agent in the first blood sample of the cat;
determining a first level of elimination of the contrast agent in the first blood sample of the cat based on a first difference between the initial concentration of the contrast agent and the first subsequent concentration of the contrast agent in the first blood sample of the cat; and
correlating the first level of elimination of contrast agent in the first blood sample of the cat with the first level of the GFR indicator at the first point in time.

4. The method of claim 3, wherein sampling at least one second level of the GFR indicator of the cat at the at least one second point in time comprises:
after at least one select second time interval, at the at least one second point in time, taking at least one second blood sample of the cat and measuring at least one second subsequent concentration of the contrast agent in the at least one second blood sample of the cat;
determining at least one second level of elimination of the iodinated nonionic radio contrast agent in the at least one second blood sample of the cat based on at least one second difference between the first subsequent concentration of the contrast agent in the first blood sample of the cat and the at least one second subsequent concentration of the contrast agent in the at least one second blood sample of the cat; and
correlating the at least one second level of elimination of the contrast agent in the at least one second blood sample of the cat with at least one second level of the GFR indicator at the second point in time.

5. The method of claim 3, wherein the contrast agent comprises an iodinated, non-ionic, radio contrast agent.

6. The method of claim 3, wherein the contrast agent comprises iohexol.

7. The method of claim 3, wherein the initial concentration of the dose of the contrast agent is in a range of about 0.5 mL/Kg to about 4.5 mL/Kg of body weight of the cat.

8. The method of claim 3, wherein the contrast agent comprises a diluting agent.

9. The method of claim 8, wherein the diluting agent comprises a saline solution.

10. The method of claim 1, wherein the GFR indicator comprises plasma clearance of iohexol.

11. A method of assessing an effectiveness of a treatment protocol for affecting kidney function in a cat comprising:
providing a plurality of cats for a control group and a plurality of cats for a treatment group;
at a first point in time, measuring a first level of a GFR indicator and calculating a baseline GFR based on the first level of the GFR indicator in each of the plurality of cats in the control group and in each of the plurality of cats in the treatment group;
pairing each of the plurality of cats in the treatment group with a corresponding cat in the plurality of cats in the control group based on a substantial similarity in the baseline GFR;
after calculation of the baseline GFR, administering to each of the plurality of cats in the treatment group a treatment composition according to the treatment protocol specifying a treatment dose and a treatment dosing interval;
at a select assessment time period at at least one second point in time following administering the treatment composition, measuring at least one second level of the GFR indicator and calculating at least one second GFR based on at least one second level of the GFR indicator in each of the plurality of cats in the control group and in each of the plurality of cats in the treatment group;
determining an evolution of a level of a kidney function over time based on a difference between the baseline GFR and the at least one second GFR over the select assessment time period for each of the plurality of cats in the control group and for each of the plurality of cats in the treatment group;
assessing the effectiveness of the treatment protocol for affecting kidney function in the cat based on a comparison of the evolution of the level of the kidney function over time in each of the plurality of cats in the treatment group with the evolution of the level of the kidney function over time in the corresponding cat in the plurality of cats in the control group;
wherein the baseline GFR of each of the plurality of cats in the treatment group and each of the plurality of cats in the control group is less than the baseline GFR of a healthy cat of a same gender.

12. The method of claim 11, wherein the select assessment time period is in a range of 45 days to 120 days.

13. The method of claim 11, wherein the select assessment time period is a minimum of approximately 60 days.

14. The method of claim 11, wherein a lack of change in the evolution of the level of the kidney function over time in the cat is correlated with a decrease in death of a plurality of cells in a glomerulus of at least one kidney in the cat.

15. The method of claim 11, wherein a positive change in the evolution of the level of the kidney function over time in the cat is correlated with a positive change in functionality of a plurality of cells in a glomerulus of at least one kidney in the cat.

16. The method of claim 11, further comprising identifying a treatment protocol for a mammal other than the cat based on an extrapolation to the mammal of the treatment protocol for affecting kidney function in the cat.

17. The method of claim 16, wherein the mammal is a human.

18. The method of claim 11, wherein measuring the first level of the GFR indicator at the first point in time comprises:
administering a dose having an initial concentration of a contrast agent to each of the plurality of cats in the control group and in the treatment group;
after a select first time interval, at the first point in time, taking a first blood sample and measuring a first subsequent concentration of the contrast agent in the first blood sample of each of the plurality of cats in the control group and in the treatment group;
determining a first level of elimination of contrast agent in the first blood sample based on a first difference between the initial concentration of the contrast agent and the first subsequent concentration of the contrast agent in the first blood sample of each of the plurality of cats in the control group and in the treatment group; and
correlating the first level of elimination of the contrast agent in the first blood sample with the first level of the GFR indicator at the first point in time in each of the plurality of cats in the control group and in the treatment group.

19. The method of claim 18, wherein measuring the at least one second level of the GFR indicator at the at least one second point in time comprises:
after the at least one select second time interval, at the at least one second point in time, taking at least one second blood sample and measuring at least one second subsequent concentration of the iodinated non-ionic contrast agent in the at least one second blood sample of each of the plurality of cats of the control group and of the treatment group;
determining at least one second level of elimination of contrast agent in the at least one second blood sample based on at least one second difference between the first subsequent concentration of the contrast agent in the first blood sample and the at least one second subsequent concentration of the contrast agent in the at least one second blood sample of each of the plurality of cats in the control group and in the treatment group; and
correlating the at least one second level of elimination of the contrast agent in the at least one second blood sample with the at least one second level of the GFR indicator at the second point in time in each of the plurality of cats in the control group and in the treatment group.

20. The method of claim 11, wherein the contrast agent comprises an iodinated nonionic radio contrast agent.

21. The method of claim 11, wherein the contrast agent comprises iohexol.

22. The method of claim 11, wherein the initial concentration of the dose of the contrast agent is in a range of about 0.5 mL/Kg to about 4.5 mL/Kg of body weight of the cat.

23. The method of claim 11, wherein the contrast agent comprises a diluting agent.

24. The method of claim 23, wherein the diluting agent comprises a saline solution.

25. The method of claim 11, wherein the GFR indicator comprises plasma clearance of iohexol.
